SEQUENCE LISTING (19 sequences)

SEQ ID NO: 1 - protein, 574 aa, Rhodococcus sp.
MVDGNYSVASNVMVPMRDGVRLAVDLYRPDADGPVPVLLVRNPYDKFDVFAWSTQSTNWLEFVRDGYAVVIQDTRGLFASEGEFVPHVDDEADAEDTLSWILEQAWCDGNVGMFGVSYLGVTQWQAAVSGVGGLKAIAPSMASADLYRAPWYGPGGALSVEALLGWSALIGRQLITSRSDARPEDAADFVQLAAICNDVAGAASVTPLAEQPLLGRLIPWVIDQVVDHPDNDESWQSISLFERLGGLATPALITAGWYDGFVGESLRTFVAVKDNADARLVVGPWSHSNLTGRNADRKFGCAATYPIQEATTMHKAFFDRHLRGETDALAGVPKVRLFVMGIDEWRDETDWPLPDTAYTPFYLGGSGAANTSTGGGTLSTSISGTESADTYLYDPADPVPSLGGTLLFHNGDNGPADQRPIHDRDDVLCYSTEVLTDPVEVTGTVSARLFVSSSAVDTDFTAKLVDVFPDGRAIALCDGIVRMRYRETLVNPTLIEAGEIYEVAIDMLATSNVFLPGHRIMVQVSSSNFPKYDRNSNTGGVIAREQLEEMCTAVNRIHRGPEHPSHIVLPIIKR

SEQ ID NO: 2 - DNA, 28 bp, synthetic construct
  source 1..28: mol_type = other DNA
ctctcataggtcgccagctcatcacgtc

SEQ ID NO: 3 - DNA, 28 bp, synthetic construct
  source 1..28: mol_type = other DNA
gacgtgatgagctggcgacctatgagag

SEQ ID NO: 4 - DNA, 25 bp, synthetic construct
  source 1..25: mol_type = other DNA
ctcgcagcaatttgcaatgacgtcg

SEQ ID NO: 5 - DNA, 25 bp, synthetic construct
  source 1..25: mol_type = other DNA
cgacgtcattgcaaattgctgcgag

SEQ ID NO: 6 - DNA, 26 bp, synthetic construct
  source 1..26: mol_type = other DNA
ggaagttcggctgcgccgcgacctac

SEQ ID NO: 7 - DNA, 26 bp, synthetic construct
  source 1..26: mol_type = other DNA
gtaggtcgcggcgcagccgaacttcc

SEQ ID NO: 8 - DNA, 32 bp, synthetic construct
  source 1..32: mol_type = other DNA
atgtgggcatgttcggcttttcgtacttgggt

SEQ ID NO: 9 - DNA, 32 bp, synthetic construct
  source 1..32: mol_type = other DNA
acccaagtacgaaaagccgaacatgcccacat

SEQ ID NO: 10 - DNA, 22 bp, synthetic construct
  source 1..22: mol_type = other DNA
gtgttcttgtggtcgacgcagt

SEQ ID NO: 11 - DNA, 22 bp, synthetic construct
  source 1..22: mol_type = other DNA
actgcgtcgaccacaagaacac

SEQ ID NO: 12 - DNA, 30 bp, synthetic construct
  source 1..30: mol_type = other DNA
ttcgacgtgttctactggtcgacgcagtcg

SEQ ID NO: 13 - DNA, 30 bp, synthetic construct
  source 1..30: mol_type = other DNA
cgactgcgtcgaccagtagaacacgtcgaa

SEQ ID NO: 14 - DNA, 23 bp, synthetic construct
  source 1..23: mol_type = other DNA
gacgtgttcgtttggtcgacgca

SEQ ID NO: 15 - DNA, 23 bp, synthetic construct
  source 1..23: mol_type = other DNA
tgcgtcgaccaaacgaacacgtc

SEQ ID NO: 16 - DNA, 32 bp, synthetic construct
  source 1..32: mol_type = other DNA
ccatacgacaagttcgacgtgttcttctggtc

SEQ ID NO: 17 - DNA, 32 bp, synthetic construct
  source 1..32: mol_type = other DNA
gaccagaagaacacgtcgaacttgtcgtatgg

SEQ ID NO: 18 - DNA, 32 bp, synthetic construct
  source 1..32: mol_type = other DNA
ccatacgacaagttcgacgtgttctggtggtc

SEQ ID NO: 19 - DNA, 32 bp, synthetic construct
  source 1..32: mol_type = other DNA
gaccaccagaacacgtcgaacttgtcgtatgg

(19) United States
(12) Patent Application Publication
(10) Pub. No.: US 2024/0117329 A1
(43) Pub. Date: Apr. 11, 2024

(54) COCAINE ESTERASE MUTANT AND USE THEREOF

(71) Applicant: Hangzhou Normal University, Hangzhou (CN)

(72) Inventors: Xiabin CHEN, Hangzhou (CN); Jianzhuang YAO, Hangzhou (CN); Shurong HOU, Hangzhou (CN); Xingyu DENG, Hangzhou (CN); Yun ZHANG, Hangzhou (CN); Junsen TONG, Hangzhou (CN)

(21) Appl. No.: 18/489,816

(22) Filed: Oct. 18, 2023

Related U.S. Application Data
(63) Continuation of application No. PCT/CN2022/089839, filed on Apr. 28, 2022.

(30) Foreign Application Priority Data
Jul. 5, 2021 (CN) 202110755649.4

(51) Int. Cl. C12N 9/18; B09C 1/10; C02F 3/34

(52) U.S. Cl. CPC C12N 9/18; B09C 1/10; C02F 3/342; C12Y 301/01084; C02F 2101/38

(57) ABSTRACT

Disclosed are a cocaine esterase mutant and use thereof. The cocaine esterase mutant is obtained by mutating a wildtype cocaine esterase, an amino acid sequence of the wildtype cocaine esterase is shown as SEQ ID No.1, the cocaine esterase mutant is T172R/G173Q/L196C/I301C, or additionally added with V116K point mutation, or additionally added with A51 site mutation, and the A51 site mutation is L, Y, V, F or W. Catalytic efficiency of the cocaine esterase mutant screened on a cocaine toxic metabolite benzoylecgonine is greatly improved compared with that of a wildtype enzyme.

### CROSS-REFERENCE TO RELATED APPLICATIONS

This application is a continuation of International Patent Application No. PCT/CN2022/089839 with a filing date of Apr. 28, 2022, designating the United States, now pending, and further claims priority to Chinese Patent Application No. 202110755649.4 with a filing date of Jul. 5, 2021. The content of the aforementioned applications, including any intervening amendments thereto, are incorporated herein by reference.

### SEQUENCE LISTING

The present application contains a sequence listing which was filed electrically in XML format and is hereby incorporated by reference in its entirety. The XML-format sequence listing file was created on Oct. 16, 2023, named as “SEQUENCE LISTING”, and is 18.428 kb in size.

### TECHNICAL FIELD

The present invention relates to the technical field of biomedicine, in particular to a cocaine esterase mutant and use thereof.

### BACKGROUND

Cocaine abuse and addiction is a serious medical and social problem in modern society. So far, no drug has been approved for cocaine detoxification treatment. The disastrous medical and social consequences of cocaine abuse make the development of anti-cocaine drugs a top priority. In vivo, about 40% of cocaine is rapidly bio-converted by butyrylcholinesterase (BChE) and carboxyesterase-2 (CE-2) to inactive metabolite ecgonine methyl ester (EME), while 45% of cocaine is hydrolyzed by carboxyesterase-1 (CE-1) in liver to toxic metabolite benzoyllecgonine (BZE) (FIG. 1). BZE has similar physiological activity to cocaine, and BZE is considered to be the main contributor to the long-term toxicity of cocaine due to its long half-life in vivo. In addition, BZE is one of the main residual pollutants of addictive drugs in the environment, which has an important impact on the ecosystem.

The ideal treatment of cocaine abuse requires not only the rapid elimination of the toxity of cocaine, but also the elimination of the toxity of the toxic metabolite BZE of cocaine. Based on the research strategy of combining computer aided design with enzyme engineering, scientists have designed and developed a series of highly efficient and heat-stable cocaine metabolizing enzymes, including human butyrylcholinesterase (BChE) mutants and bacterial cocaine esterase (CocE) mutants, which can effectively eliminate the toxicity of cocaine (Larsen N A, Turner J M, Stevens J, Rosser S J, Basran A, Lerner R A, Bruce N C, Wilson I A. Crystal structure of a bacterial cocaine esterase, Nat Struct Biol 2002, 9 (1): 17-21.). On this basis, further degradation of BZE is the key to complete detoxification of cocaine, so it is of great significance to develop efficient metabolizing enzymes for BZE degradation for complete detoxification of cocaine.

Endogenous BChE is the only one that can hydrolyze BZE into non-toxic ecgonine (ECG) and benzoic acid. However, due to the high glycosylation of natural BChE, efficient and economical recombinant expression of BChE has always been a difficult problem in the industry, which is difficult in later development. Therefore, it is of great application value to find BZE metabolizing enzymes with economic expression and high catalytic activity.

CocE may be economically and efficiently expressed by a prokaryotic cell expression system, and the recombinant protein has good safety in human (the CocE T172R/G173Q mutant has completed the clinical test II phase of cocaine detoxification, and proved that the mutant is safe and effective). Nasser, A. F., et al. J. Addit. Dis. 2014, 33, 289-302). Therefore, CocE is a very ideal candidate BZE metabolizing enzyme. However, the catalytic efficiency of natural CocE to BZE is too low, and a great improvement is needed to meet the requirement of efficiently removing the toxicity of BZE.

### SUMMARY

The present invention aims to design and obtain a brand-new high-activity CocE mutant to further improve the catalytic activity of a cocaine toxic metabolite BE, may be used for clinical treatment of cocaine intoxication, and meet the clinical use requirement.

Based on the catalytic mechanism of phenyl ester hydrolysis by esterase and the classical catalytic triad structure of esterase, we found cocaine esterase as a candidate enzyme for hydrolyzing BZE by searching through Rosetta software. We found for the first time that CocE could catalyze BZE hydrolysis to give ecgonine and benzoic acid, with BZE catalytic parameters as follows: k_{cat}=301.2 min⁻¹, and K_{M}=5153 μM.

A cocaine esterase mutant is obtained by mutating a wildtype cocaine esterase, wherein an amino acid sequence of the wildtype cocaine esterase is shown as SEQ ID No.1, and
the cocaine esterase mutant is one of the following:
(1) V116K,
(2) T172R/G173Q/V116K,
(3) T172R/G173Q/L196C/I301C/V116K,
(4) T172R/G173Q/L196C/1301C/V116K/A51L,
(5) T172R/G173Q/L196C/1301C/V116K/A51Y,
(6) T172R/G173Q/L196C/1301C/V116K/A51V,
(7) T172R/G173Q/L196C/1301C/V116K/A51F, and
(8) T172R/G173Q/L196C/1301C/V116K/A51W.

The present invention further provides use of the cocaine esterase mutant for preparing a medicament for treating cocaine intoxication.

The present invention further provides use of the cocaine esterase mutant for preparing a hydrolytic agent for hydrolyzing benzoylecgonine into ecgonine and benzoic acid.

The present invention further provides use of the cocaine esterase mutant for treating cocaine or benzoylecgonine contamination in water or soil.

The present invention also provides a medicament for treating cocaine intoxication, wherein an active ingredient is the cocaine esterase mutant.

The medicament further comprises a medically acceptable additive.

The present invention also provides a reagent for treating cocaine or benzoylecgonine contamination in water or soil, wherein an effective ingredient is the cocaine esterase mutant.

The present invention also provides a gene encoding the cocaine esterase mutant.

The present invention also provides an expression vector containing the gene.

The present invention also provides a recombinant expression cell containing the expression vector.

The present invention also provides use of the gene according for treating cocaine or benzoylecgonine contamination in water or soil.

The present invention obtains a series of mutations through computer aided design, and constructs a CocE mutant by adopting a point mutation PCR method; expresses and purifies the recombinant protease; and verifies the enzyme activity by in vitro and in vivo enzymatic reactions, and screens high-activity mutants.

According to the research of the present invention, the catalytic efficiency of the V116K mutant obtained by mutating the 116′ V (valine) of cocaine esterase into K (lysine) on the cocaine toxic metabolite benzoylecgonine is greatly improved compared with the wildtype enzyme. On the basis of the mutation of V116K, screening is further carried out, and the catalytic efficiency of the screened cocaine esterase mutant on the cocaine toxic metabolite benzoylecgonine is further improved.

### BRIEF DESCRIPTION OF THE DRAWINGS

FIG. 1 is a metabolic pathway of cocaine.

FIG. 2 is a technical roadmap of the present invention.

FIG. 3A and FIG. 3B are graphs showing a high-activity CocE mutant accelerating BZE metabolism in rats: FIG. 3A is a curve of a BEZ concentration in blood over time; and FIG. 3B is a curve of a metabolite BA concentration in blood over time; wherein, 5M-51L represents T172R/G173Q/L196C/I301C/V116K/A51L; and 5M-51V represents T172R/G173Q/L196C/I301CN116K/A51V.

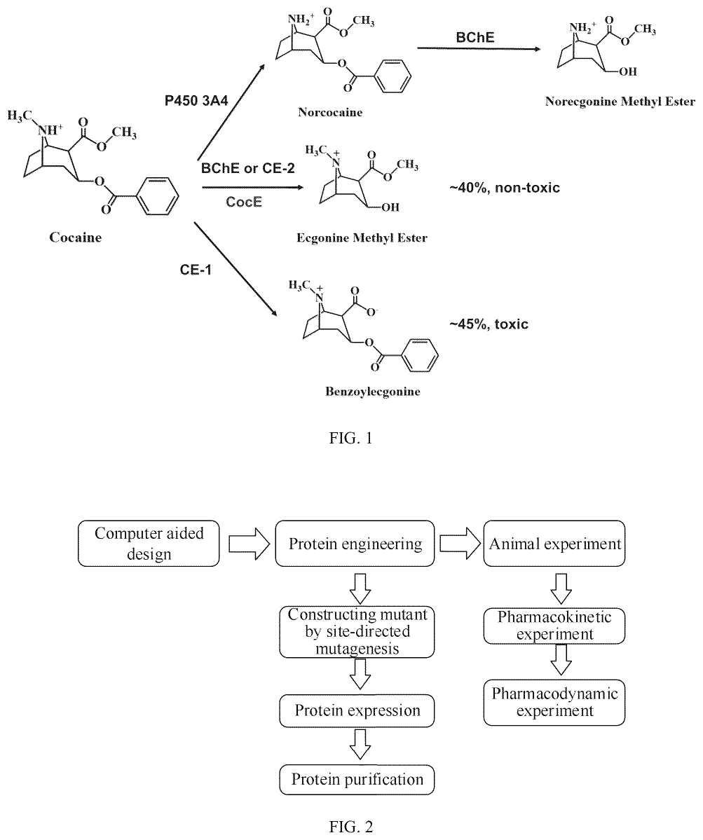

### DETAILED DESCRIPTION

The overall technical route of the present invention is shown in FIG. 2.

### Embodiment 1: Site-Directed Mutagenesis

CocE mutants were obtained by site-directed mutagenesis.

Firstly, cDNA (GenBank #AF173165.1, synthesized by Shanghai Generay Biotech) of wildtype CocE was constructed into an Escherichia coli expression vector pET-22b (+) (provided by Shanghai Generay Biotech, and a target gene containing C-terminal-6×His was inserted into NdeI and XhoI restriction site). Wildtype CocE plasmid was used as a template to design mutant primers, PCR products of the mutants were obtained through PCR (KOD One Mater Mix, Shanghai Toyobo) amplification. DNA templates in the products were removed through DpnI (Thermo Scientific, FD1703), and then transformed into competent cells (DH5a) to cyclize the PCR products. The transformed competent cell bacterial solution was coated on LB solid medium containing 100 μg/ml ampicillin, and cultured at 37° C. for 15 hours. Monoclones were selected and the mutant plasmids were extracted by plasmid extraction kit. The mutant plasmids with correct sequence were confirmed by DNA sequencing. For those with multiple mutations, one mutation was followed by next round of mutation. Mutant primer design was shown in table 1.

TABLE 1
Primer sequences used for site-directed mutagenesis

| Mutation site | Primer (5′-3′) | |
|---|---|---|
| T172R/G173Q | Forward primer | CTCTCATAGGTCGCCAGCTCATCACGTC |
| | Reverse primer | GACGTGATGAGCTGGCGACCTATGAGAG |
| L196C | Forward primer | CTCGCAGCAATTTGCAATGACGTCG |
| | Reverse primer | CGACGTCATTGCAAATTGCTGCGAG |
| I301C | Forward primer | GGAAGTTCGGCTGCGCCGCGACCTAC |
| | Reverse primer | GTAGGTCGCGGCGCAGCCGAACTTCC |
| V116K | Forward primer | ATGTGGGCATGTTCGGCTTTTCGTACTTGGGT |
| | Reverse primer | ACCCAAGTACGAAAAGCCGAACATGCCCACAT |
| A51L | Forward primer | GTGTTCTTGTGGTCGACGCAGT |
| | Reverse primer | ACTGCGTCGACCACAAGAACAC |
| A51Y | Forward primer | TTCGACGTGTTCTACTGGTCGACGCAGTCG |
| | Reverse primer | CGACTGCGTCGACCAGTAGAACACGTCGAA |
| A51V | Forward primer | GACGTGTTCGTTTGGTCGACGCA |
| | Reverse primer | TGCGTCGACCAAACGAACACGTC |
| A51F | Forward primer | CCATACGACAAGTTCGACGTGTTCTTCTGGTC |
| | Reverse primer | GACCAGAAGAACACGTCGAACTTGTCGTATGG |
| A51W | Forward primer | CCATACGACAAGTTCGACGTGTTCTGGTGGTC |
| | Reverse primer | GACCACCAGAACACGTCGAACTTGTCGTATGG |

### Embodiment 2: Protein Expression and Purification

The successfully constructed mutant plasmids were transformed into Escherichia coli BL21 competent cells to express proteins, the bacterial solution was inoculated into an LB liquid medium (containing 100 μg/ml ampicillin), subjected to enlarge cultivation on a shaker at 37° C. and 250 rpm until OD₆₀₀=0.6-0.8, and then the bacterial solution was cooled to 15° C. IPTG (Sigma, 367-93-1) was added until a final concentration was 1 mM, and then protein expression was induced at 15° C. and 180 rpm for 15 hours. The cells were collected and re-suspended in 50 mM Tris-HCl buffer (pH 7.4) containing 150 mM NaCl. Escherichia coli cells were disrupted with a precooled high-pressure cell disrupter (SCIENTZ JG-IA, Ningbo Scientz), centrifuged at 9,000 rpm for 45 minutes and then the supernatant was collected. The supernatant was mixed with a cobalt medium (Takara, TALON Metal Affinity Resin) by rotation at 4° C. for 2 hours to bind the protein containing 6×His to the medium. The binding solution was added into a gravity column, naturally flowing out under the action of gravity, and the target protein was purified by using a gradient elution method of imidazole with different concentrations. The eluted components were collected in a 30K (Millipore) ultrafiltration tube, and the buffer was replaced by centrifugal concentration. The protein was stored in solution S (50 mM HEPES, 20% D-sorbitol, 1 M glycine, pH 7.4). Protein concentration was determined by Bradford kit (Sangon Biotech, C503031-1000).

### Embodiment 3: Enzyme Activity Analysis

Firstly, an experimental method for detecting a substrate BZE and a product benzoic acid BA by HPLC was established. Both BZE and BA had strong ultraviolet absorption at 230 nm. In HPLC analysis, acetonitrile and 0.1% formic acid were used as mobile phases to separate BZE and BA by C18 liquid chromatography column. The absorption of BZE and BA at wavelength 230 nm was detected by an ultraviolet detector, and linear standard curves of BZE and BA were obtained. The activity of BZE reaction catalyzed by CocE was determined, and the reaction temperature was 25° C., with three repetitions in each group. An enzymatic reaction was started by 50 μL of substrate BZE solution with 50 μL of enzyme solution (diluted with 0.1 M phosphate buffer (pH 7.4)). The specific reaction conditions were shown in Table 2.

TABLE 2
Conditions for in vitro catalytic reaction of
high-activity mutant enzyme to substrate BZE

| | BZE | Reaction time |
|---|---|---|
| Enzyme and concentration thereof | concentration(μM) | (min) |
| Wildtype, | 100, 250 | 4 |
| T172R/G173Q, | 500, 1,000, 2,500, | 10 |
| T172R/G173Q/L196C/I301C, (50 nM) | 5,000, 7,500, 12,500 | |
| T172R/G173Q/L196C/I301C/V116K, | 5, 10, 20, 50, 100, 200 | 2 |
| T172R/G173Q/L196C/I301C/V116K/A51L, | 500, 1,000 | 5 |
| T172R/G173Q/L196C/I301C/V116K/A51Y, (4 nM) | | |
| V116K, | 5, 10, 20, 50, 100, 200 | 4 |
| T172R/G173Q/V116K, | 500, 1,000 | 10 |
| T172R/G173Q/L196C/I301C/V116K/A51V, | | |
| T172R/G173Q/L196C/I301C/V116K/A51F, | | |
| T172R/G173Q/L196C/I301C/V116K/A51W, (4 nM) | | |

50 μL of 10% perchloric acid was added to stop the reaction, and then, 50 μL of acetonitrile was added, and the mixture was centrifuged at 12,000 rpm for 5 minutes. Then, the supernatant was diluted to an appropriate multiple and injected into 100 L. The peak time, peak areas and curves of BZE and BA were compared, and the residual BZE concentration and the BA concentration in the reaction sample were calculated. The k_{cat} and K M of each mutant could be obtained by calculating the reaction rates of BA catalyzed by enzyme at different substrate concentrations, drawing a kinetic curve of the enzyme reaction with GraphPad Prism 8, and performing Michaelis-Menten kinetic analysis. The results were shown in Table 3.

TABLE 3
Catalytic kinetic parameters of high-activity mutant enzyme to substrate BZE

| | K_{M} | k_{cat} | K_{eff} | |
|---|---|---|---|---|
| Mutation site | (μM) | (min⁻¹) | (M⁻¹min⁻¹) | RCE |
| Wildtype WT | 5,153 | 301.2 | 5.85 × 10⁴ | 1.0 |
| V116K | 89.57 | 320.4 | 3.58 × 10⁶ | 61.2 |
| T172R/G173Q | 4,355 | 418.7 | 9.61 × 10⁴ | 1.6 |
| T172R/G173Q/V116K | 65.63 | 425.2 | 6.48 × 10⁶ | 110.8 |
| T172R/G173Q/L196C/I301C | 3,709 | 556.7 | 1.50 × 10⁵ | 2.6 |
| T172R/G173Q/L196C/I301C/V116K | 46.26 | 568.6 | 1.23 × 10⁷ | 210.3 |
| T172R/G173Q/L196C/I301C/V116K/A51L | 27.84 | 873 | 3.14 × 10⁷ | 536.5 |
| T172R/G173Q/L196C/I301C/V116K/A51Y | 43.59 | 910.6 | 2.09 × 10⁷ | 357.4 |
| T172R/G173Q/L196C/I301C/V116K/A51V | 86.16 | 693.2 | 8.05 × 10⁶ | 137.6 |
| T172R/G173Q/L196C/I301C/V116K/A51F | 59.29 | 733.9 | 1.24 × 10⁷ | 211.8 |
| T172R/G173Q/L196C/I301C/V116K/A51W | 95.64 | 740.7 | 7.74 × 10⁶ | 132.5 |

Note:
K_{eff} refers to the catalytic efficiency (k_{cat}/K_{M}) of the corresponding enzyme to the substrate BZE.
RCE refers to a ratio of the catalytic efficiency of the mutant enzyme to BZE and that of the wildtype enzyme to BZE.

### Embodiment 4: In Vivo Experiments in Animals

Experimental male SD rats (200 g/rat) purchased from Laboratory Animal Center of Zhejiang Academy of Medical Sciences were raised in a constant-temperature and constant-humidity environment. The feeding and experimental application was carried out by following Guide for the Care and Use of Laboratory Animals.

(1) Standard Curves for BA and BZE Blood Samples

Blood was collected from the femoral vein of rats using a blood collection needle and a heparin-treated capillary. Firstly, eight tubes of blood from the same rat, 75 μL in each tube, were added to 100 μL of 250 μM paraxon to inhibit the effect of endogenous metabolizing enzymes on BZE, and frozen at −80° C. After thawing, 19.7 μL of mixed solution containing 0, 4, 10, 20, 40, 60, 100 and 200 μM BA and BE standards, then subjected to vortex for 20 seconds, and then added with 150 μL of acetonitrile and subjected to vortex for 1 minutes, then added with 50 μL of 10% HClO₄ and subjected to vortex for 1 minutes, centrifuged at 17,000 rcf for 15 minutes, and then centrifuged again after the supernatant was transferred. 250 μL of supernatant were taken out for HPLC analysis. The sample volume was 100 μL, and the HPLC experimental conditions were the same as those in Embodiment 3. The concentrations of BZE and BA in the final standard samples were 0, 0.2, 0.5, 1, 2, 3, 5 and 10 μM respectively.

(2) High-Activity CocE Mutant Accelerated BZE Metabolism In Vivo

There were 5 rats in each group. Firstly, 0.2 or 1 mg/kg of high-active CocE mutant or normal saline was injected into the tail vein of rats, and 2 mg/kg BZE was injected into the tail vein within 1 minute. After BZE injection, 75 μL of blood samples were taken at the 0^{th}, 2^{nd}, 5^{th}, 10^{th}, 30^{th}, 60^{th}, 90^{th} and120^{th} minutes respectively, and 100 μL of 250 μM paraxon were added in each sample, and then frozen at −80° C. After thawing, the samples were subjected to vortex for 20 seconds, then added with 150 μL of acetonitrile and subjected to vortex for 1 minute, then added with 50 μL of 10% HClO₄ and subjected to vortex for 1 minute, centrifuged at 17,000 g for 15 minutes, and then centrifuged again after the supernatant was transferred. 250 μL of supernatant were taken out for HPLC analysis. The sample volume was 100 μL. The BA and BZE concentrations in the blood of rats in each group at different time points were calculated according to the blood sample standard curve of the standards, so as to obtain the metabolism of BZE with or without the high-activity CocE mutant.

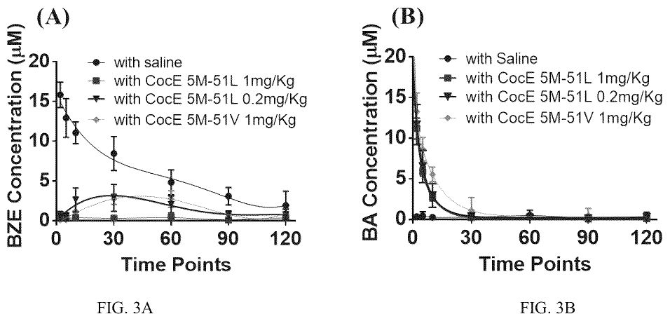

The results were shown in FIG. 3A and FIG. 3B. The results showed that after intravenous injection of 2 mg/kg of BZE in the rats, the BZA concentration in the blood was as high as 15.8 μM and the BA concentration in the blood was less than 0.5 μM at the second minutes. When the high-activity mutant was injected, the BZA concentration in the blood of rats decreased rapidly while the BA concentration in the blood increased rapidly, which indicated that the toxic BZE in the rats was quickly eliminated by the high-activity mutant and metabolized into non-toxic BA.

1. A cocaine esterase mutant obtained by mutating a wildtype cocaine esterase, wherein an amino acid sequence of the wildtype cocaine esterase is shown as SEQ ID No.1, and
the cocaine esterase mutant is one of the following:
(1) V116K,
(2) T172R/G173Q/V116K,
(3) T172R/G173Q/L196C/1301C/V116K,
(4) T172R/G173Q/L196C/1301C/V116K/A51L,
(5) T172R/G173Q/L196C/1301C/V116K/A51Y,
(6) T172R/G173Q/L196C/1301C/V116K/A51V,
(7) T172R/G173Q/L196C/1301C/V116K/A51F, and
(8) T172R/G173Q/L196C/1301C/V116K/A51W.

2. The cocaine esterase mutant according to claim 1, wherein the cocaine esterase mutant is used for preparing a medicament for treating cocaine intoxication.

3. The cocaine esterase mutant according to claim 1, wherein the cocaine esterase mutant is used for preparing a hydrolytic agent for hydrolyzing benzoylecgonine into ecgonine and benzoic acid.

4. The cocaine esterase mutant according to claim 1, wherein the cocaine esterase mutant is used for treating cocaine or benzoylecgonine contamination in water or soil.

5. A gene encoding the cocaine esterase mutant according to claim 1.

6. The gene according to claim 5, wherein the gene is used for treating cocaine or benzoylecgonine contamination in water or soil.

7. An expression vector containing the gene according to claim 5.

8. A recombinant expression cell containing the expression vector according to claim 6.
